(19) United States
(12) Patent Application Publication
(10) Pub. No.: US 2021/0259995 A1
(43) Pub. Date: Aug. 26, 2021

(54) METHODS FOR INHIBITING GROWTH OF ACSL4-OVEREXPRESSING TUMORS

(71) Applicant: CONSEJO NACIONAL DE INVESTIGACIONES CIENTÍFICAS Y TÉCNICAS (CONICET), Ciudad Autónoma de Buenos Aires (AR)

(72) Inventors: Ernesto Jorge PODESTÁ, Ciudad Autónoma de Buenos Aires (AR); Ana Fernanda CASTILLO, Ciudad Autónoma de Buenos Aires (AR); Ulises Daniel ORLANDO, Monte Grante Pcia. de Buenos Aires (AR); Paula Mariana MALOBERTI, Ciudad Autónoma de Buenos Aires (AR); Angela Rosaria SOLANO, Ciudad Autónoma de Buenos Aires (AR)

(21) Appl. No.: 17/313,639

(22) Filed: May 6, 2021

Related U.S. Application Data
(62) Division of application No. 16/348,432, filed on May 8, 2019.
(60) Provisional application No. 62/419,013, filed on Nov. 8, 2016.

(51) Int. Cl. A61K 31/15; A61P 35/00; A61K 31/337; A61K 31/4439; A61K 31/704; A61K 45/06

(52) U.S. Cl. CPC A61K 31/15; A61P 35/00; A61K 45/06; A61K 31/4439; A61K 31/704; A61K 31/337

(57) ABSTRACT

Compositions and methods for inhibiting tumor growth, particularly breast cancer, in a combined pharmacological treatment. A pharmaceutical combination for inhibiting growth of a tumor overexpressing ACSL4, comprising a first and a second dosage forms. The first dosage form has an ACSL4 inhibitor and the second dosage form has a chemotherapeutic agent.

### FIELD OF THE INVENTION

The present invention relates to compositions and methods for preventing and treating proliferative diseases, such as cancer. More specifically, the present invention relates to compositions and methods for inhibiting tumor growth, particularly breast cancer, in a combined pharmacological treatment. Yet more specifically, the method of the present invention is a method for inhibiting growth of ACSL4-overexpressing tumors. In a preferred embodiment, the invention provides a pharmaceutical composition for inhibiting tumor growth comprising a combination of: i) a first component which is an ACSL4 inhibitor; and ii) a second component selected from a group of chemotherapeutics drugs.

### BACKGROUND OF THE INVENTION

The design of cancer chemotherapy has become increasingly sophisticated, yet there is no cancer treatment that is 100% effective against disseminated cancer, i.e., metastatic cancer. Resistance to treatment with anticancer drugs results from a variety of factors including individual variations in patients and somatic cell genetic differences in tumors, even those from the same tissue of origin. Frequently, resistance is intrinsic to the cancer, but as therapy becomes more and more effective, acquired resistance has also become common. The most common reason for acquisition of resistance to a broad range of anticancer drugs is expression of one or more energy-dependent transporters that detect and eject anticancer drugs from cells, yet other mechanisms of resistance—including insensitivity to drug-induced apoptosis and induction of drug-detoxifying mechanisms—probably play an important role in acquired anticancer drug resistance. Drug inactivation can also play a major role in the development of resistance. These mechanisms include, for example, conjugation of the drug to GSH, a powerful anti-oxidant that protects the cells against the damaging effects of reactive oxygen species.

Studies on mechanisms of cancer drug resistance have yielded important information about how to circumvent this resistance to improve cancer chemotherapy. Also, these mechanisms of cancer drug resistance have been shown to affect the pharmacokinetics of many commonly used drugs.

The present inventors have previously shown that the acyl-CoA synthetase 4 (ACSL4), an enzyme taking part in arachidonic acid metabolism, plays a key role in the hormone resistance to therapy and in the mechanism of breast cancer aggressiveness, particularly in metastatic breast cancer (MBC) [Maloberti P M, Duarte A B, Orlando U D, Pasqualini M E, Solano A R, Lopez-Otin C and Podesta E J Functional interaction between acyl-CoA synthetase 4, lipooxygenases and cyclooxygenase-2 in the aggressive phenotype of breast cancer cells. PLoS One. 2010; Orlando U D, Garona J, Ripoll G V, Maloberti P M, Solano A R, Avagnina A, Gomez D E, Alonso D F and Podesta E J. The functional interaction between Acyl-CoA synthetase 4, 5-lipooxygenase and cyclooxygenase-2 controls tumor growth: a novel therapeutic target. PLoS One. 2012; Orlando U D, Castillo A F, Dattilo M A, Solano A R, Maloberti P M and Podesta E J. Acyl-CoA synthetase-4, a new regulator of mTOR and a potential therapeutic target for enhanced estrogen receptor function in receptor positive and -negative breast cancer. Oncotarget. 2015]. Consequently, the aim of the present application was to study the role of ACSL4 on mechanisms of cancer drug resistance. Herein, and by means of the stable transfection of MCF-7 cells with ACSL4 using the tetracycline Tet-Off system (MCF-7 Tet-Off/ACSL4), ACSL4-responsive drug resistance genes were identified, that were significantly and differentially expressed in MCF-7 Tet-Off/ACSL4 compared to MCF-7 Tet-Off empty vector.

Furthermore, it is shown herein that the minimal doses of the chemotherapeutic agents, doxorubicin and paclitaxel which produced a significant inhibition on cell proliferation was significantly higher in the ACSL4 expressing cells. It is shown that ACSL4 inhibitors Triacsin C and rosiglitazone can act in combination with the chemotherapeutic agents to inhibit cell growth. In addition, a synergistic effect on cell growth inhibition by these combinations is demonstrated. Remarkably, this synergistic effect is also evident in the triple negative MDA-MB-231 cells in vitro and in vivo. These results suggest that ACSL4 proves to be a target for restoring tumor drug sensitivity in tumors with poor prognosis for disease-free and overall survival, in which no effective specifically targeted therapy is readily available.

The treatment of metastatic cancer has become increasingly aimed at molecular targets derived from studies of the oncogenes and tumor suppressors known to be involved in the development of human cancers.

A study of the mechanisms by which cancers elude treatment has yielded a wealth of information about why these therapies fail and is beginning to yield valuable information about how to evade drug resistance in cancer cells and/or design agents that are not subject to the usual means of resistance.

Tumor heterogeneity may also contribute to resistance, where small subpopulations of cells may acquire or stochastically already possess some of the features enabling them to emerge under selective drug pressure. Making the problem even more challenging, some of these resistance pathways lead to multidrug resistance, generating an even more difficult clinical problem to overcome.

A great deal is now known about mechanisms of drug resistance in cancer cells. Despite the development of new targeted anticancer therapies, mechanisms that have evolved in mammals to protect cells against cytotoxic compounds in the environment will continue to act as obstacles to successful treatment of cancer. Additional knowledge about these mechanisms of cancer drug resistance may help to design strategies to circumvent resistance and new drugs that are less susceptible to known resistance mechanisms.

Nevertheless, resistance to drugs continues to be a major problem in oncology affecting the majority of cancer patients. Therefore, the design of anti-cancer drugs that are fully effective necessitates a better understanding of the mechanisms by which cancer cells elude treatment.

Metastatic breast cancer (MBC) is generally considered to be incurable, with response rates and duration of response progressively declining with subsequent lines of treatment. MBC is a heterogeneous disease and is among the leading causes of cancer mortality, accounting for more than 400,000 deaths annually worldwide.

Tumors are either intrinsically resistant to systemic therapy or acquire resistance at some point during multiple courses of therapy. Targeted anticancer agents for the treatment of breast cancer, such as hormonal agents or the more recently approved epidermal growth factor receptor (EGFR) inhibitor, are also associated with intrinsic and acquired resistance.

A variety of strategies have been devised to prevent or overcome resistance to systemic anticancer therapy, including drug combinations and sequential regimens. However, it appears that resistance to established cytotoxic and targeted agents is inevitable. Novel agents with reduced susceptibility to resistance may prevent or delay the emergence of resistance and improve survival in patients with common solid tumors, including metastatic breast cancer. There is a hope that further elucidation of the cellular and molecular processes that allow tumor cells to develop resistance and the use of new agents to combat these mechanisms will improve outcomes for patients with metastatic breast cancer.

Triple-negative breast cancer (estrogen-receptor-α (ER)-negative, progesterone-receptor (PR)-negative, and human epidermal growth factor 2 receptor (HER2)-non overexpressed) is a subtype of breast cancer that accounts for approximately 15% of breast cancer. Triple-negative breast cancer (TNBC) is a subtype of tumor known for its aggressive clinical behavior.

Triple negative breast cancer and endocrine-resistant breast cancer tumors are an important area of research for both researchers and clinicians alike for being poor prognostic factors for disease-free and overall survival. Besides, no effective specific targeted therapy is readily available therefor.

For the treatment of triple-negative breast cancer, there is renewed interest in investigating the role of chemotherapeutic agents, however still there is a significant toxic effect of this drug.

The most “classic” drug combinations explored to date in the clinical disease settings are only additive or, at most, minimally synergistic. The traditional approach for the introduction of new agents has been to add the drug to accepted and/or established regimens. Since the leading goal of combination therapy is to attain therapeutic effectiveness and the reduction of adverse side effects, the search continues for the ideal drug partners that will act in this way to maximize tumor responses while offsetting tumor progression.

In the present application the inventors demonstrate that the sensitivity of different breast cancer cell lines to chemotherapy agent correlates inversely with the expression of acyl-CoA synthetase 4 (ACSL4). Unlike the other ACSL isoforms, ACSL4 is encoded on the X chromosome and its expression is highest in adrenal cortex, ovary and testis (Kang, M. J. et al., 1997. A novel arachidonate-preferring acyl-CoA synthetase is present in steroidogenic cells of the rat adrenal, ovary, and testis. Proc Natl Acad Sci USA 94, 2880-2884.). ACSL4 is also highly expressed in mouse and human cerebellum and hippocampus. The physiological functions of ACSL4 have been studied and include possible roles in polyunsaturated fatty acid metabolism in brain, in steroidogenesis and in eicosanoid metabolism related to apoptosis. ACSL4 expression has also been associated with non-physiological functions such as mental retardation disorder (Modi, H. R. et al., 2013. Propylisopropylacetic acid (PIA), a constitutional isomer of valproic acid, uncompetitively inhibits arachidonic acid acylation by rat acyl-CoA synthetase 4: a potential drug for bipolar disorder. Biochim Biophys Acta 1831, 880-886.) and cancer (Maloberti P. M. et al., 2010, supra). ACSL4 was first associated with cancer due to its abnormal expression in colon and hepatocellular carcinoma. Increased ACSL4 expression, both at mRNA and protein levels, in colon adenocarcinoma cells has been associated with the inhibition of apoptosis and an increase in cell proliferation when compared to adjacent normal tissue. The effect of conventional chemotherapeutic agents was analyzed in terms of the proliferative capacity of cells expressing ACSL4 vs cells that did not express ACSL4, using the MCF-7 Tet-Off/ACSL4 and MCF-7 Tet-Off/empty vector. The specificity of ACSL4 was established by the specific inhibition of its expression in doxycycline-treated MCF-7 Tet-Off/ACSL4 cells in which the doses that produced a significant inhibition in cell proliferation were similar to the effect observed with the MCF-7 Tet-Off/empty vector cells.

Referring again to the mechanisms of drug resistance, it is generally accepted that failure of a patient's cancer to respond to a specific therapy can result from one of two general causes: host factors and specific genetic or epigenetic alterations in the cancer cells. Host factors include poor absorption or rapid metabolism or excretion of a drug, resulting in low serum levels; poor tolerance to side effects of a drug, especially in elderly patients, reducing the side effects by means of reduction of the dose would not result in a therapeutic effect; inability to deliver a drug to the site of a tumor, as could occur with bulky tumors or with biological agents of high molecular weight and low tissue penetration such as monoclonal antibodies and immunotoxins; and various alterations in the host-tumor environment that affect response of the tumor including local metabolism of a drug by non-tumor cells, unusual features of the tumor blood supply that may affect transit time of drugs within tumors and the way in which cells in a cancer interact with each other and with interstitial cells from the host.

It came as something of a surprise that the major mechanism of multidrug resistance in cultured cancer cells was the expression of an energy-dependent drug efflux pump, known as the multidrug transporter. This efflux pump, the product of the MDR1 gene in the human and the product of two different, related genes, mdr1a and mdr1b in the mouse, was one of the first members described of a large family of ATP-dependent transporters known as the ATP-binding cassette (ABC) family. ABC proteins transport various molecules across extra- and intra-cellular membranes. ABC genes are divided into seven distinct subfamilies (ABC1, MDR/TAP, MRP, ALD, OABP, GCN20, White). The membrane-associated protein encoded by ABCG2 gene is included in the superfamily of ATP-binding cassette (ABC) transporters. This protein is a member of the White subfamily. Alternatively referred to as a breast cancer resistance protein, this protein functions as a xenobiotic transporter which may play a major role in multi-drug resistance. It likely serves as a cellular defense mechanism in response to mitoxantrone and anthracycline exposure. The protein encoded by the ABCC8 gene is also a member of the superfamily of ATP-binding cassette (ABC) transporters. In particular, this protein is a member of the MRP subfamily which is involved in multi-drug resistance. This protein functions as a modulator of ATP-sensitive potassium channels. In turn, the protein encoded by the ABCC4 gene is a member of the ABC1 subfamily. Members of the ABC1 subfamily comprise the only major ABC subfamily found exclusively in multicellular eukaryotes. (Gottesman, M. M. (2002) Mechanisms of cancer drug resistance. Annu Rev Med. 53: p. 615-27; Zahreddine, H, Borden, K. L. (2013) Mechanisms and insights into drug resistance in cancer. Front Pharmacol. 4: p. 28). Every living organism has encoded within its genome many members of this family, and they appear to be involved not only in the efflux of drugs but also in moving nutrients and other biologically important molecules into, out of, and across plasma membranes and intracellular membranes in cells.

By up or down regulation of the expression of ACSL4 in several cell lines, the present inventors demonstrate that ACSL4 expression regulates the expression of several genes of the MDR (energy-dependent transporters) in breast cancer cells.

Combination drug regimens with newer cytotoxic and biologic therapies are an effective strategy in fighting tumor growth and/or progression. These combinations can facilitate the attack on multiple intercellular processes, which may result in more efficient tumor responses. These strategies may also delay or circumvent mechanisms of drug resistance by interfering with cell survival or tumor growth pathways and the cross-talk established between them. Chemotherapeutic agents alone may become ineffective because other alternative pathways support tumor survival.

Here the present inventors demonstrate that the combination of inhibition of ACSL4 activity with a chemotherapy agent produces a strong significant effect in vitro that reduces tumor growth in a triple negative human breast cancer cell line. It is remarkable that the doses used of both agents, an ACSL4 inhibitor and a chemotherapy agent did not produce any effect per se which is an important finding that lead the present inventors to suggest the probability of decreasing side toxic effects of these agents when used in effective doses. The strong significant effect observed by the present inventors is achieved together with a reduction in the doses which also avoids adverse and toxic side effects by the chemotherapeutic agents, also allowing the combination therapy to cover a wide spectrum of signaling pathways which support tumor cells survival.

Although drug combinations are often used to treat early breast cancer, advanced breast cancer is more often treated with single chemotherapeutic drugs. Still, some combinations, such as carboplatin or cisplatin plus gemcitabine are commonly used to treat advanced breast cancer.

The majority of current combination regimens have been developed empirically and although patterns of cross-resistance, overlapping drug toxicity, and mechanisms of action are considered when designing such regimens, formal preclinical testing has played only a minor role.

One attempt to minimize the impact of drug resistance has been the concurrent use of two or more chemotherapeutic agents with unrelated mechanisms of action and differing modes of drug resistance, with the intent of blocking the development of multiple intracellular escape pathways, which are essential for tumor survival. Within the past decade, an array of mechanistically diverse agents has augmented the list of combination regimens that may be both synergistic and efficacious in pretreated MBC. However, these combination regimens have not rendered the expected results.

In the study herein the present inventors demonstrate that the inhibition of ACSL4 activity may be a very powerful strategy to be combined with chemotherapeutic agents to reduce side effects by reducing the individual effective doses of these agents, due to the strong significant effect that can be observed when the inhibition of ACSL4 activity is part of the combined therapy. This has demonstrated that both drugs contribute and work in a concerted manner to increase the inhibitory effect on tumor cell proliferation.

The present inventors have demonstrated a positive correlation of ACSL4 expression and aggressiveness in breast cancer cell lines, with the highest expression found in metastatic lines derived from triple-negative tumor breast cancer (MDA-MB-231 and Hs578T) (Maloberti et. al., 2010). Functionally, it was found that ACSL4 is part of the mechanism responsible for increased breast cancer cell proliferation, invasion and migration, both in vitro and in vivo (Maloberti et al., supra, 2010; Orlando et al., 2012, supra). Accordingly, the sole transfection of MCF-7 cells, a model of non-aggressive breast cancer cells, with ACSL4 cDNA transforms them into a highly aggressive phenotype, and it was further demonstrated that ACSL4 can be silenced to reduce cell line aggressiveness. Furthermore, the stable transfection of MCF-7 cells with ACSL4 using the tetracycline Tet-Off system (MCF-7 Tet-Off/ACSL4) and their injection into nude mice has resulted in the development of growing tumors with marked nuclear polymorphism, a high mitotic index and low expression of ER and PR (Orlando et al., 2012, supra), all of which demonstrates the transformational capacity of ACSL4 overexpression. The role of ACSL4 in the development of growing tumors found further support when tumor growth was inhibited through the inhibition of ACSL4 expression by treating mice with doxycycline. Although the role of ACSL4 in mediating the aggressive phenotype in breast cancer is well accepted, the mechanism involved in this effect has yet to be fully elucidated. And, as enzyme overexpression can solely change cell phenotype from mildly aggressive to highly aggressive, the MCF-7 Tet-Off/ACSL4 model may be regarded as a valuable technique to study the mechanisms through which ACSL4 triggers the phenotype change.

In conclusion, ACSL4 is an upstream regulator of tumorigenic pathways and the data herein provide novel insights into a combined pharmacological approach. Because an aggressive tumor phenotype appears in the early stages of metastatic progression, the previously unknown mediators of ACSL4 might become valuable prognostic tools or therapeutic targets in breast cancer.

In addition, the present inventors have shown in previous studies that the effects of Rosiglitazone on cell and tumor growth in vitro or in vivo are similar to those obtained with the specific inhibition of ACSL4 by doxycycline treatment of the MCF-7 Tet-Off/ACSL4 (Orlando et al., 2012, supra), the minimal doses of rosiglitazone exerting significant inhibitory effects being 75 μM.

Rosiglitazone is an antidiabetic drug in the thiazolidinedione class of drugs. It works as an insulin sensitizer, by binding to the PPAR receptors in fat cells and making the cells more responsive to insulin. Despite rosiglitazone's effectiveness at decreasing blood sugar in type 2 diabetes mellitus, at daily oral dose in the range of 4 to 8 mg, its use decreased dramatically as studies showed apparent associations with increased risks of heart attacks and death. On Sep. 23, 2010 the US Food and Drug Administration issued a decision to restrict access to rosiglitazone medicines. In Europe, the European Medicines Agency (EMA) recommended in September 2010 that the drug be suspended from the European market because the benefits of rosiglitazone no longer outweighed the risks.

Rosiglitazone, a member of the thiazolidinedione family of drugs (TZDs), is known to attenuate cell growth in carcinoma of various organs including breast, prostate, lung, colon, stomach, bladder and pancreas. Rosiglitazone and derivatives of troglitazone have been used either alone or in combination in experimental conditions to inhibit the growth of different tumor cell lines (Luconi, M et al., 2010. Rosiglitazone impairs proliferation of human adrenocortical cancer: preclinical study in a xenograft mouse model. Endocr Relat Cancer 17, 169-177) and although the action of rosiglitazone has been attributed to its effects on the peroxisome proliferator-activated receptor gamma, in vitro studies performed with rat recombinant proteins have demonstrated that TZDs can directly inhibit the activity of one of the gene products of the acyl-CoA synthetases, i.e. ACSL4.

Another member of the drug class of thiazolidinediones, Troglitazone, a peroxisome proliferator-activated receptor gamma agonist, which enhances insulin sensitivity, was approved for the treatment of type 2 diabetes in 1997. Troglitazone was available in 400 mg tablets. The recommended dosage was 400 to 800 mg once daily. However, within a year after its widespread use, individual cases of liver injury and failure were reported, leading to the withdrawal of troglitazone from the market in the year 2000.

Another of the compounds used by the inventors in the combinations proposed in the present invention is Triacsin C (N-(((2E,4E,7E)-undeca-2,4,7-trienylidene)amino)nitrous amide). Triacsin C was discovered by Yoshida K, and other Japanese scientists, in 1982, in a culture of the microbe Streptomyces aureofaciens. They identified it as a vasodilator. Triacsin C belongs to a family of fungal metabolites all having an 11-carbon alkenyl chain with a common N-hydroxytriazene moiety at the terminus. Due to the N-hydroxytriazene group, triacsin C has acidic properties and may be considered a polyunsaturated fatty acid analog.

Triacsin C blocks β-cell apoptosis, induced by fatty acids (lipoapoptosis) in a rat model of obesity. In addition, it blocks the de novo synthesis of triglycerides, diglycerides, and cholesterol esters, thus interfering with lipid metabolism. Particularly Triacsin C is an inhibitor of ACSL1 and ACSL4.

As was previously mentioned herein, triple-negative breast cancer (TNBC) is highly aggressive, resulting in poor prognosis. Chemotherapy of TNBC relies on anti-cancer agents with strong cytotoxicity, but it causes several side effects with recurrence. While combinational approaches of chemotherapeutics have been highlighted as a new treatment strategy for TNBC to reduce side effects, combinations of anti-cancer agents with non chemotherapeutic drugs have been poorly reported. The present inventors show herein that newly combined drugs inhibit TNBC growth. Considering a combinational strategy for TNBC treatment, we further studied strongly significant effects of inhibitors of ACSL4 activity with various anti-cancer drugs in TNBC treatment.

The chemotherapeutic agent doxorubicin, which is a cytotoxic anthracycline antibiotic isolated from Streptomyces peuxetus, has been widely used in the clinical treatment of a broad spectrum of cancers. The mechanism underlying the antitumor effect of Doxorubicin has been associated with its ability to induce the apoptosis of cancer cells. Unfortunately, Doxorubicin exhibits cytotoxic effects on a wide range of cells, as well as cancer cells. In addition, Doxorubicin may be fatal in animals as it damages several organs, including the heart, bones and kidneys. The clinical application of Doxorubicin has been limited due to cardiomyopathy and heart failure associated with Doxorubicin usage. The severity of cardiac damage is typically proportional to the cumulative dose of Doxorubicin in a patient (Singal P K, Iliskovic N Doxorubicin-induced cardiomyopathy. N Engl J Med. 1998; 339: 900-9058). Therefore, it is not possible to increase the antitumor potency of doxorubicin by increasing the dose of doxorubicin due to its adverse effects.

Breast cancer is one of the most common types of cancer and is the fourth leading cause of cancer-associated mortality worldwide. Since the 1970s, Doxorubicin has been considered one of the most effective agents for the treatment of advanced breast cancer. However, recent studies have demonstrated that numerous cancer cell types, including breast cancer cells, are resistant to the apoptosis-inducing effects of Doxorubicin. Therefore, the identification of sensitizing agents that are able to increase the potency of Doxorubicin at low doses with clinically acceptable adverse effects may help to improve the treatment of breast cancer.

In recent years, numerous strategies were proposed to reverse Doxorubicin resistance. An important method to overcome Doxorubicin resistance is to use two or more chemotherapeutic drugs with different anti-tumor mechanisms (Zhao et al. 2016. Doxorubicin and resveratrol co-delivery nanoparticle to overcome doxorubicin resistance. Scientific Reports. 6:35267. DOI: 10.1038/srep35267). However, the pharmacokinetic properties of different drugs are vastly different, Therefore, just using two or more free chemotherapeutic drugs can not effectively overcome Doxorubicin resistance or enhance the anticancer effects of the drugs.

Taxanes are a class of diterpenes. They were originally identified from plants of the genus Taxus (yews), and feature a taxadiene core. Paclitaxel (Taxol) and docetaxel (Taxotere) are widely used as chemotherapy agents.

The principal mechanism of action of the taxane class of drugs is the disruption of microtubule function. Microtubules are essential to cell division, and taxanes stabilize GDP-bound tubulin in the microtubule, thereby inhibiting the process of cell division as depolymerization is prevented.

Cremophor EL (CrEL-) paclitaxel (Taxol), initially approved for the treatment of relapsed ovarian cancer, received US Food and Drug Administration (FDA) approval in 1994 for the treatment of patients with MBC who did not respond to anthracycline-based combination chemotherapy or with breast cancer that recurred within 6 months of adjuvant chemotherapy. (Rowinsky E K, Donehower R C. Paclitaxel (taxol) N Engl J Med. 1995; 332(15): 1004-14; Cortazar P, Justice R, Johnson J, Sridhara R, Keegan P, Pazdur R. US Food and Drug Administration approval overview in metastatic breast cancer. J Clin Oncol. 2012; 30(14): 1705-11) Approval was based on a phase III trial of 2 different doses (175 or 135 mg/m²). Similarly, docetaxel was used at a dose of 100 mg/m².

Both CrEL-paclitaxel and docetaxel have demonstrated significant clinical efficacy in MBC; however, both agents are associated with characteristic toxicities, mainly hypersensitivity reactions and peripheral neuropathy at least partially due to their respective solvents—CrEL and polysorbate 80.

A number of combination therapies have been studied for the treatment of metastatic breast cancer, and several taxane combinations are highlighted by the National Comprehensive Cancer Network (NCCN) as preferred regimens, including doxorubicin with docetaxel or CrEL-paclitaxel, capecitabine with docetaxel, and gemcitabine with CrEL-paclitaxel.

Therefore, digging into the early steps through which ACSL4 increases tumor growth and progression, the present inventors have recently demonstrated the role of ACSL4 in tumor insensitivity to hormone therapy [Orlando et al., 2015, supra]. However, and even if the role of ACSL4 in mediating an aggressive phenotype in breast cancer is well accepted, its involvement in drug resistance mechanisms has not been determined yet.

In this context, the present application provides a new combination useful for inhibiting growth of ACSL4-overexpressing tumors as well as for those drug-resistant tumors, particularly, triple negative breast cancer.

### SUMMARY OF THE INVENTION

It is an object of the present invention to provide a pharmaceutical combination for inhibiting growth of a tumor overexpressing ACSL4, comprising a first and a second dosage forms, wherein: i) the first dosage form comprises an ACSL4 inhibitor; and ii) the second dosage form comprises a chemotherapeutic agent.

According to a preferable embodiment the ACSL4 inhibitor is present in a sub-therapeutically effective amount in the first dosage form.

According to a preferably embodiment the chemotherapeutic agent is present in a sub-therapeutically effective amount in the second dosage form.

It is an object of the present invention to provide a pharmaceutical composition for inhibiting growth of a tumor overexpressing ACSL4, comprising: i) an ACSL4 inhibitor; and ii) a chemotherapeutic agent.

According to a preferred embodiment the ACSL4 inhibitor is present in a sub-therapeutically effective amount in the composition of the invention.

According to a preferred embodiment the chemotherapeutic agent is present in a sub-therapeutically effective amount in the composition of the invention

In accordance with one aspect of this invention, the pharmaceutical combination is effective for inhibiting tumor growth, the tumor overexpressing ACSL4, particularly the tumor is selected from the group consisting of colon carcinoma, hepatocellular carcinoma, prostate cancer, breast cancer, triple negative breast cancer (TNBC).

In another aspect, the pharmaceutical composition is effective for inhibiting tumor growth, the tumor overexpressing ACSL4, particularly the tumor is selected from the group consisting of colon carcinoma, hepatocellular carcinoma, prostate cancer, breast cancer, triple negative breast cancer (TNBC).

According to one aspect the tumor overexpressing ACSL4 is metastatic.

More particularly the triple negative breast cancer (TNBC) is metastatic.

According to the invention, the ACSL4 inhibitor is selected from the group of Triacsin C, and a thiazolidinedione. The thiazolidinedione is preferably selected from the group of rosiglitazone, pioglitazone and troglitazone, being rosiglitazone the most preferred.

According to the invention, the chemotherapeutic agent is selected from doxorubicin, and a taxane.

According to another preferred embodiment the chemotherapeutic agent is a taxane selected form paclitaxel, albumin-bound paclitaxel and docetaxel.

In a preferred embodiment, the pharmaceutical combination of the invention comprises a first and a second dosage forms, wherein: i) the first dosage form comprises a ACSL4 inhibitor selected from the group of Triacsin C, and a thiazolidinedione, and ii) the second dosage form comprises a taxane, wherein the taxane may be selected from paclitaxel, albumin-bound paclitaxel and docetaxel.

In another preferred embodiment, the pharmaceutical combination of the invention comprises a first and a second dosage forms, wherein: i) the first dosage form comprises Triacsin C, and ii) the second dosage form comprises an anthracycline or a derivative thereof, which can be doxorubicin, daunorubicin, idarubicin, morpholinodoxorubicin, morpholinodaunorubicin, methoxymorpholinyldoxorubicin, or derivatives or combinations thereof. Most preferably, the anthracycline is doxorubicin.

According to another embodiment, the pharmaceutical composition of the invention comprises: i) an ACSL4 inhibitor selected from the group of Triacsin C, and a thiazolidinedione, and ii) a taxane, wherein the taxane may be selected from paclitaxel, albumin-bound paclitaxel and docetaxel.

In another preferred embodiment, the pharmaceutical composition of the invention comprises: i) Triacsin C, and ii) an anthracycline or a derivative thereof, which can be doxorubicin, daunorubicin, idarubicin, morpholinodoxorubicin, morpholinodaunorubicin, methoxymorpholinyldoxorubicin, or derivatives or combinations thereof. Most preferably, the anthracycline is doxorubicin.

The thiazolidinedione is preferably selected from the group of rosiglitazone, pioglitazone and troglitazone, being rosiglitazone the most preferred.

According to a most preferred embodiment the pharmaceutical combination comprises a dosage form comprising Triacsin C and a dosage form comprising doxorubicin.

According to a most preferred embodiment the pharmaceutical combination comprises a dosage form comprising Triacsin C and a dosage form comprising paclitaxel.

According to a most preferred embodiment the pharmaceutical combination comprises a dosage form comprising rosiglitazone and a dosage form comprising doxorubicin.

According to a most preferred embodiment the pharmaceutical combination comprises a dosage form comprising rosiglitazone and a dosage form comprising paclitaxel.

According to another most preferred embodiment, the pharmaceutical composition comprises Triacsin C and doxorubicin.

According to another most preferred embodiment the pharmaceutical composition comprises Triacsin C and paclitaxel.

According to another most preferred embodiment the pharmaceutical composition comprises rosiglitazone and doxorubicin.

According to another most preferred embodiment the pharmaceutical composition comprises rosiglitazone and paclitaxel.

In another embodiment, the present invention provides a method for treating a patient having a tumor overexpressing ACSL4, the method comprising administering to the patient: i) an ACSL4 inhibitor; and ii) a chemotherapeutic agent.

According to a preferred embodiment the ACSL4 inhibitor is administered according to a sub-therapeutically effective amount.

According to a preferred embodiment the chemotherapeutic agent is administered according to a sub-therapeutically effective amount.

According to the invention the tumor is colon carcinoma, hepatocellular carcinoma, prostate cancer, breast cancer or triple negative breast cancer (TNBC).

According to one aspect the tumor overexpressing ACSL4 is metastatic.

More particularly the triple negative breast cancer (TNBC) is metastatic.

According to a preferred embodiment, the method comprises administration to a patient having a tumor overexpressing ACSL4: i) an ACSL4 inhibitor selected from the group of Triacsin C, and a thiazolidinedione, and ii) a chemotherapeutic agent which is a taxane. The taxane may be selected from paclitaxel, albumin-bound paclitaxel and docetaxel.

According to another preferred embodiment, the method comprises administration to a patient having a tumor overexpressing ACSL4: i) an ACSL4 inhibitor selected from the group of Triacsin C and a thiazolidinedione, and ii) a chemotherapeutic agent which is an anthracycline or a derivative thereof, which can be doxorubicin, daunorubicin, idarubicin, morpholinodoxorubicin, morpholinodaunorubicin, methoxymorpholinyldoxorubicin, or derivatives or combinations thereof. Most preferably, the agent is doxorubicin.

In one aspect of the method the thiazolidinedione compound is preferably selected from the group of rosiglitazone, pioglitazone and troglitazone, being rosiglitazone the most preferred.

In one aspect of the method the ACSL4 inhibitor is selected from the group of Triacsin C, and rosiglitazone, and the chemotherapeutic agent is selected from doxorubicin, paclitaxel and docetaxel.

According to a preferred embodiment of the method the ACSL4 inhibitor is Triacsin C, and the chemotherapeutic agent is selected from paclitaxel and docetaxel.

According to a preferred embodiment of the method the ACSL4 inhibitor is Triacsin C, and the chemotherapeutic agent is doxorubicin.

According to another aspect of the method the ACSL4 inhibitor is rosiglitazone, and the chemotherapeutic agent is selected from paclitaxel and docetaxel.

According to a preferred embodiment of the method the ACSL4 inhibitor is rosiglitazone, and the chemotherapeutic agent is doxorubicin.

In another aspect of the invention the method comprises administering to a patient in need thereof an amount of an ACSL4 inhibitor prior, o during, administering an amount of the chemotherapeutic agent.

### BRIEF DESCRIPTION OF THE DRAWINGS

The following figures form part of the present specification and are included to further illustrate certain aspects of the present invention.

FIG. 1 shows the protein expression of multidrug resistance genes in ACSL4-overexpressing cells using Western blot analysis. Data is presented as fold change. Genes analyzed were ABCG2, ABCC4 and ABCC8. Whole cell extracts were obtained as previously described [Maloberti P M et. al., 2010] from MCF-7 Tet-Off empty vector, MCF-7 Tet-Off/ACSL4 and doxycycline-treated MCF-7 Tet-Off/ACSL4 cells (Doxy, 1 μg/ml, 48 h). Western blot was performed using the indicated antibodies. Representative blots are shown (upper panels). The integrated optical density of protein levels was quantified and normalized with the corresponding β-tubulin signal (lower panels). A: ABCG2, B: ABCC4 and C: ABCC8. Data represent the means±SD of three independent experiments. a: ***p<0.001 vs. MCF-7 Tet-Off empty vector cells and b: ***p<0.001 vs. MCF-7 Tet-Off/ACSL4 cells.

FIG. 2 shows the effect of ACSL4 on protein expression of multidrug resistance genes in MDA-MB-231 cells using Western blot analysis. A) MDA-MB-231 cells were transfected with the pSUPER.retro plasmid containing a shRNA targeted to ACSL4 (shRNA-ACSL4) or an empty plasmid (mock) as previously described ((Maloberti P. M. et al., 2010, supra). One month post selection, whole cell extracts were obtained and analyzed by Western blot as previously described using the indicated antibodies. Data represent the means of fold changes (shRNA-ACSL4 vs. mock)±SD of three independent experiments. ***p<0.001 vs. control cells. B) MDA-MB-231 cells were incubated with Triacsin C (luM) or rosiglitazone (100 uM). Forty-eight hours post-treatment, whole cell extracts were obtained and analyzed by Western blot as previously described using the indicated antibodies. Representative blots (upper panels) and integrated optical density of protein levels (lower panels) quantified and normalized with the corresponding β-tubulin signal are shown. Data represent the means of fold changes Inh-ACSL4 vs. mock)±SD of three independent experiments. ***p<0.001 vs. control cells.

FIG. 3 shows ACSL4 expression on cell proliferation inhibition by chemotherapeutic agents using the MCF-7 Tet-Off model. MCF-7 Tet-Off empty vector and MCF-7 Tet-Off/ACSL4 cells were plated at a density of 4000 cells/well in 96-well plates with 10% FBS-supplemented D-MEM and allowed to adhere overnight at 37° C. in a humidified 5% CO₂ atmosphere. The medium was then changed to serum-free medium. After 24 h, the cells were switched to 10% FBS-supplemented D-MEM with A. doxorubicin (0.025-0.100 μM) and B. paclitaxel (0.1-0.5 μM), and with doxycycline (0.025 μM) where indicated. After 48 h, cell proliferation was measured by BrdU incorporation assay. Data are presented as inhibition of cell proliferation compared to control cells. Data are presented as the mean±SD. *p<0.05, **p<0.01 and ***p<0.001 MCF-7 Tet-Off empty vector cells vs. MCF-7 Tet-Off/ACSL4 cells; a, b and c: p<0.001 MCF-7 Tet-Off/ACSL4 doxycycline-treated cells vs. MCF-7 Tet-Off/ACSL4 cells.

FIG. 4 shows cell proliferation inhibition by combining sub-effective doses of ACSL4 inhibitor rosiglitazone and chemotherapeutic agents in MDA MB-231 cells. MDA-MB-231 cells were plated as describe in FIG. 3 and then incubated with rosiglitazone (20 μM) and/or A. doxorubicin (0.025 μM), B. paclitaxel (0.5 μM) for 96 h. Subsequently, cell proliferation was measured by BrdU incorporation assay. Data are presented as inhibition of cell proliferation compared to control cells. White bars indicate single inhibitor treatment while grey bars indicate combined inhibitor treatment. Data are presented as the mean±SD. a, b: ***p<0.001 vs. corresponding single inhibitors.

FIG. 5 shows cell proliferation inhibition by combining sub-effective doses of ACSL4 inhibitor Triacsin C and chemotherapeutic agents in MDA MB-231 cells. MDA-MB-231 cells were plated as described in FIG. 3 and then incubated with Triacsin C (0.01-0.10 μM) and/or A. doxorubicin (0.025 μM), B. paclitaxel (0.1 μM) for 96 h. Subsequently, cell proliferation was measured by BrdU incorporation assay. Data are presented as inhibition of cell proliferation compared to control cells. White bars indicate single inhibitor treatment while black bars indicate combined inhibitor treatment. Data are presented as the mean±SD. **p<0.01, ***p<0.001 vs. corresponding single inhibitors.

FIG. 6 shows the inhibition on cell migration by combining sub-effective doses of ACSL4 inhibitor rosiglitazone and chemotherapeutic agents in MDA MB-231 cells. Wound healing assay was performed as described in Materials and Methods section. Cells were incubated with rosiglitazone (20 μM) and/or A. doxorubicin (0.025 and 0.05 μM), B. paclitaxel (0.5 and 1.0 μM). At the specified time points, the distance between the wound edges was measured using Image-Pro Plus software. Data represent the means±SD of three independent experiments. A) 4 h-assay: a p<0.05 vs Control, b p<0.05 vs Rosiglitazone, c p<0.05 vs Doxorubicin 0.025 mM, d p<0.05 vs control, e p<0.05 vs Rosiglitazone, f p<0.05 vs Doxi 0.05; 8 h-assay: g p<0.001 vs control, h p<0.001 vs Rosiglitazone, i p<0.001 vs Doxorubicin 0.025, j p<0.001 vs control, k p<0.001 vs Rosiglitazone, l p<0.001 vs Doxorubicin 0.05. B) a p<0.001 vs Control, b p<0.001 vs Rosiglitazone, c p<0.001 vs Paclitaxel 1 mM, d p<0.001 vs control, e p<0.001 vs Rosiglitazone, f p<0.001 vs Paclitaxel 0.5 mM, g p<0.0011 vs control, h p<0.001 vs Rosiglitazone, i p<0.001 vs Paclitaxel 1.0 mM.

FIG. 7 shows the effect of ACSL4 inhibition in combination with doxorubicin in the MDA-MB-231 human breast cancer xenograft model. Mice bearing MDA-MB-231 tumor xenografts were treated with either vehicle (control), rosiglitazone, doxorubicin alone, or in a combination of two drugs as indicated for 30 consecutive days. Comparison of A. average tumor volume, and B. tumor growth rate between days 20-30 was determined. Data are presented as the mean±SD, n=5. Significant differences by one-way ANOVA, A: a p<0.001 vs Control, b p<0.001 vs Rosiglitazone, c p<0.001 vs Doxorubicin; B: a p<0.001 vs Control, b p<0.001 vs Rosiglitazone, c p<0.001 vs Doxorubicin.

### DETAILED DESCRIPTION OF THE INVENTION

The present application discloses that ACSL4 overexpression is a novel regulator of drug resistance genes; therefore, the combined inhibition of an upstream mechanism such as ACSL4 activity seems to be a potential target to be used in order to avoid compensatory feedback. Besides, as ACSL4 has been related to colon and hepatocellular carcinoma, besides breast carcinoma, the present findings suggest novel mediators, specifically for combined pharmacological treatment toward tumor growth inhibition.

By common usage, the term chemotherapy has come to connote the use of rather non-specific intracellular poisons, especially related to inhibiting the process of cell division known as mitosis. Traditional chemotherapeutic agents are cytotoxic by means of interfering with cell division (mitosis) but cancer cells vary widely in their susceptibility to these agents. To a large extent, chemotherapy can be thought of as a way to damage or stress cells, which may then lead to cell death if apoptosis is initiated.

All chemotherapy regimens require that the patient be capable of undergoing the treatment. Performance status is often used as a measure to determine whether a patient can receive chemotherapy, or whether dose reduction is required. Because only a fraction of the cells in a tumor dies with each treatment (fractional kill), repeated doses must be administered to continue to reduce the size of the tumor. Current chemotherapy regimens apply drug treatment in cycles, with the frequency and duration of treatments limited by toxicity to the patient. Dosage of chemotherapy can be difficult: If the dose is too low, it will be ineffective against the tumor, whereas, at excessive doses, the toxicity (adverse side-effects) will be intolerable to the patient.

Many of the side effects of chemotherapy can be traced to damage to normal cells that divide rapidly and are thus sensitive to anti-mitotic drugs: cells in the bone marrow, digestive tract, and hair follicles. This results in the most common side-effects of chemotherapy: myelosuppression (decreased production of blood cells, hence also immunosuppression), mucositis (inflammation of the lining of the digestive tract), and alopecia (hair loss).

Therefore it is desired that a composition for treating a tumor comprises a chemotherapeutic agent in a sub-therapeutically effective amount.

By sub-therapeutically effective amount is meant an amount that is less than the amount of the agent generally found to be clinically optimally effective when the agent is administered alone. A sub-therapeutically effective amount can also mean an amount that is less than the amount required of the agent generally found to elicit a defined clinical response. It should be appreciated that sub-therapeutically effective is not intended to mean that the agent is clinically ineffective when administered according to the present inventive compositions and methods.

In a preferred embodiment, the sub-therapeutically effective amount of the agent can be insufficient to effectively alter susceptibility to agents, particularly susceptibility of diseased cells. For example, the sub-therapeutically effective amount of the agent can be insufficient to inhibit effectively, growth, metastasis, and/or replication of tumor cells. Also, the sub-therapeutically effective amount of the agent can be insufficient to inhibit effectively an increase in the number of tumor cells in a patient having a tumor. The sub-therapeutically effective amount of the agent also can be insufficient to decrease effectively the number of tumor cells in a patient having a tumor. In addition, the sub-therapeutically effective amount of the agent can be insufficient to increase effectively mortality of tumor cells, increase effectively susceptibility of tumor cells to damage by anti-cancer agents.

### Doses

According to the present invention the compositions contain an acyl-CoA synthetase 4 (ACSL4) inhibitor selected from rosiglitazone, troglitazone or pioglitazone.

Rosiglitazone may be in an amount of about 0.01 mg to about 20 mg, more suitably in a range of about 0.1 mg to about 5 mg and more preferably in a range of about 0.5 mg to about 2 mg per dose unit. All dose units are preferably referred to body weight (approx. 70 kg). Preferably, rosiglitazone is present in the composition of the invention in an amount ranging from 2 to 8 mg/70 kg (body weight).

Troglitazone may be may be in an amount of about 1 mg to about 600 mg, more suitably in a range of about 5 mg to about 400 mg and more preferably in a range of about 50 mg to 200 mg, per dose unit. All dose units are preferably referred to body weight (approx. 70 kg).

Triacsin C is used at a dose of 8-15 mg/kg per day in monotherapy. According to one aspect of the present invention, the Triacsin C dosage used in combination therapy in vivo is 8 times lower than the minimum dose that produces a significant inhibition when administered alone. According to one aspect of the present invention, the Triacsin C dosage used in combination therapy in cell culture is 5 times lower than the minimum dose that produces a significant inhibition when administered alone.

Doxorubicin is used herein at a concentration of 0.1 μM in cell culture. The in vivo dose of doxorubicin in human patients as a monotherapy is approximately 25 mg/m² via i.v. administration. According to one aspect of the present invention, the doxorubicin dosage used in combination therapy is 2.6 times lower than the minimum dose that produces a significant inhibition when administered alone. In other words, the amount used in combination is 12 times lower than the maximum effective dose when applied alone. This means that the doxorubicin dose used in a combination produces the same inhibitory effect than the drug alone, but at a 12-times lower concentration.

Paclitaxel is used herein at a concentration of 1-5 μM in cell culture. The in vivo dose of paclitaxel in human patients as a monotherapy is approximately 135-175 mg/m² via i.v. administration every two weeks. According to another aspect of the present invention, the paclitaxel dosage used in combination therapy is half the minimum dose that produces a significant inhibition when administered alone. In other words, the amount used in combination is 5 times lower than the maximum effective dose when applied alone. This means that the paclitaxel dose used in a combination produces the same inhibitory effect than the drug alone, but at a 5-times lower concentration.

As used herein, a “pharmaceutical combination” refers to a product where two or more separate drug preparations are packaged together in a single package or as a unit. Similarly a “pharmaceutical combination” refers to two or more drug preparations packaged separately that according to its proposed labeling is for use only together. In particular, according to the present invention, each drug may be formulated with a suitable carrier/excipient thus forming separate individual preparations, in order to administer them in a simultaneous or sequential way.

As used herein, a “pharmaceutical composition” refers to a product that comprises one or more active ingredients and an optional carrier/excipient. The composition may comprise inert ingredients, as well as any product that results, directly or indirectly, from the combination, complexing or aggregation of any two or more ingredients, or from the dissociation of one or more of the ingredients, or from other types of reactions or interactions of one or more of the ingredients. In general, the pharmaceutical compositions are prepared by uniformly and intimately associating the active ingredient(s) with a liquid carrier/excipient or a finely divided solid carrier/excipient or both, and later, if desired, conforming the product in the desired formulation. In particular, according to the present invention, each active ingredient may be formulated with a suitable carrier/excipient, and after that, if desired, the formulations may be combined to form a single final preparation.

The pharmaceutical compositions of the present invention comprise any composition prepared by mixing active compound(s) and at least one pharmaceutically acceptable carrier/excipient. By “pharmaceutically acceptable”, it is meant that the carrier, diluent or excipient must be compatible with the other ingredients of the formulation and must not be harmful for its recipient.

The term “treatment” as used herein refers to any treatment of a condition or human disease and includes: (1) inhibiting the disease or condition, that is, deterring its development, (2) alleviating the disease or condition, that is, causing the regression of the condition, or (3) deterring the symptoms of the disease.

The term “to inhibit” includes its generally accepted meaning that includes “to restrict,” “to alleviate,” “to improve,” and “to slow,” “to deter or to invert the progression, severity or a resulting symptom.” As used herein, the term “therapy”, such as in “drug therapy” or in relation to any medical therapy, includes in vivo or ex vivo diagnostic and therapeutic techniques carried out in humans.

In general, the pharmaceutical compositions of the present invention can be administered by standard routes, such as by parenteral route (for example, intravenous, intravertebral, subcutaneous or intramuscular), oral, tracheal, bronchial, intranasal, pulmonary, buccal, rectal, transdermal or topical. The administration can be systemic, regional or local.

The types of pharmaceutical compositions that can be used include: tablets or pills, chewable tablets, capsules (including microcapsules), powders, powders for reconstitution, solutions, parenteral solutions, aerosol solutions, ointments (creams and gels), suppositories, suspensions, and other types described herein or that are evident for an expert in the field, from general knowledge of the art. The active principle(s), for example, can also be in the form of a complex including cyclodextrins, their ethers or esters.

The inhibitory compounds used in the present invention may be taken in suitable forms for administration by ordinary processes, using auxiliary or excipient substances such as liquid or solid ingredients, in powder, such as pharmaceutically usual liquids or solids and expanders, solvents, emulsifiers, lubricants, flavoring agents, pigments and/or buffering substances (buffers). Frequently used auxiliary or excipient substances include: magnesium carbonate, titanium dioxide, lactose, sucrose, sorbitol, mannitol and other sugars or sugar alcohols, talcum, lacto protein, gelatin, starch, amylopectin, cellulose and its derivatives; animal and vegetable oils such as fish liver oil, sunflower, peanut or sesame, polyethylene glycol; and solvents such as sterile water and mono- or polyhydric alcohols such as glycerol; as well as disintegrating agents and lubricating agents such as magnesium stearate, calcium stearate, sodium stearyl fumarate and polyethylene glycol waxes. Then, the mixture may be processed into granules or compressed into tablets.

Each active ingredient can be separately premixed with the other non-active ingredients, before being mixed to form a formulation or, alternatively, the active ingredients can be mixed together, before being mixed with the inert ingredients to form a formulation.

Soft gelatin capsules can be prepared with capsules that contain a mixture of the active ingredients of the invention, vegetable oil, fat, or other vehicles suitable for soft gelatin capsules. Hard gelatin capsules can contain granules of the active ingredients. Hard gelatin capsules can also contain the active ingredients with solid ingredients in powder, such as lactose, sucrose, sorbitol, mannitol, potato starch, cornstarch, amylopectin, cellulose derivatives or gelatin.

Units for rectal administration can be prepared (i) in the form of suppositories that contain the active substances mixed with a base of neutral fat; (ii) in the form of a rectal gelatin capsule that contains the active substance in mixture with a vegetable oil, paraffin oil or another vehicle suitable for rectal gelatin capsules; (iii) in the form of a ready-to-use micro enema; or (iv) in the form of a dry micro enema formulation to be reconstituted in a suitable solvent before its administration.

Liquid preparations can be prepared in the form of syrups, elixirs, drops or concentrated suspensions, for example, solutions or suspensions that contain the active principles and the remainder consists of for example, sugar or sugar alcohols, and a mixture of ethanol, water, glycerol, propylene glycol and polyethylene glycol, if desired, such liquid preparations can contain pigment agents, flavoring agent, preservatives, saccharin and carboxymethylcellulose and other thickening agents. Liquid preparations can also be prepared in dry powder form, reconstituted with suitable solvent before their use. Solutions for parenteral administration can be prepared as the solution of a formulation of the invention in a pharmaceutically acceptable solvent, such as a sterile water solution or non-water solvent, as vegetable oil, esters of long-chain aliphatic acids or propylene glycol. These solutions can also contain stabilizers, preservatives and/or buffers. Solutions for parenteral administration can also be prepared as a dry preparation, reconstituted with a suitable solvent before their use.

The compositions of the invention to be applied topically on the skin or the scalp can be prepared in the form of ointments (creams or gels). In an embodiment of the invention, an oil emulsion is prepared in water to form a cream. The active compounds in powder form are dissolved in a suitable solvent, such as, for example, propylene glycol. The aqueous phase can alternatively include an alcohol or isopropanol, adding a thickener, for example, Carbomer 934 or 940. The oily phase preferably includes mineral oil, petroleum jelly, cetyl alcohol and/or stearyl alcohol. Emulsifiers which can be used are: polysorbate 80, sorbitan monostearate or others known in the art. Buffering agents, antioxidants and chelating agents may also be added to improve the characteristics of the formulation.

Preparations for topical administration can be prepared for delivery in an aerosol. In these cases, the inhibitory compounds can be admixed with known excipients for aerosol, such as saline solution, alcohol, or fatty acid derivatives, to enhance bioavailability if necessary.

Formulations are also supplied in accordance with the present invention as “kits” that comprise one or more containers that separately contain one or more of the ingredients of the pharmaceutical composition of the invention in a suitable carrier/excipient, for its co-administration. These containers may include indications for the use thereof, such as instructions for use, or a notification in the form prescribed by a governmental agency that governs the manufacture, use or sale of pharmaceutical products, whose notification reflects approval by the agency of the manufacture, use or sale for human use.

The terms “combination therapy” or “co-administration” are intended to embrace the administration of each active agent in a sequential way, in a system that will provide the beneficial effects resulting from the combination of drugs, and it is intended to embrace the co-administration of these agents in a substantially simultaneous way, such as in a single dose unit that has a fixed ratio of these ingredients, or in multiple dose units, separate for each active agent.

The amount of each active ingredient and the dosage system to treat a disease condition with the compounds and compositions of the invention depends on a variety of factors, including: age, weight, sex and medical condition of the patient, severity of the disease and route and frequency of administration, as well as the particular compound employed, so that it can vary widely.

### Materials and Methods

### Materials

Dulbecco's modified Eagle medium (DMEM), penicillin-streptomycin solution and trypsin-EDTA were purchased from GIBCO, Invitrogen Corporation (Grand Island, N.Y., USA). Fetal Calf Serum was from PAA laboratories GmbH (Pasching, Austria). Doxycycline and 3-(4,5-dimethyl-2-thiazolyl)-2,5-diphenyl-2H-tetrazoliumbromide) (MTT) were purchased from Sigma Chemical Co. (St. Louis, Mo., USA). ABCG2, ABCC8 and ABCC4 antibodies were purchased from Origene technologies (Rockville Md., USA).

Horseradish peroxidase-conjugated goat anti-rabbit and goat-anti-mouse secondary antibodies, Immun-Blot polyvinylidene fluoride membrane was from Bio-Rad Laboratories (Hercules, Calif., USA). Enhanced chemiluminescence (ECL) was from GE Healthcare (Buckinghamshire, UK) and Tri-Reagent was from Molecular Research Center (Cincinnati, Ohio, USA). RNase-free Deoxyribonuclease I (DNase I) were obtained from Invitrogen (Carlsbad, Calif.). M-MLV reverse transcriptase (RT) was purchased from Promega (Madison, Wis., USA). SYBR Select Master Mix was obtained from Applied Biosystems (Carlsbad, Calif., USA). Sterile and plastic material for tissue culture was from Orange Scientific (Braine-l'Alleud, Belgium). 5-bromo-2′-deoxyuridine (BrdU) cell proliferation ELISA kit was from Roche Diagnostics. All other reagents were of the highest grade available.

### Cell Culture

Human breast cancer cell line was generously provided by Dr. Vasilios Papadoupoulus (Research Institute of the McGill University Health Centre, Montreal, Canada) and obtained from the Lombardi Comprehensive Cancer Center (Georgetown University Medical Center, Washington D.C. USA). The tetracycline-repressible MCF-7 cell lines, designated MCF-7 Tet-Off empty vector, and MCF-7 Tet-Off-induced repression of ACSL4, designated MCF-7 Tet-Off/ACSL4 were obtained previously in the laboratory (Maloberti P. M. et al., 2010).

Quantitative Reverse Transcription-PCR (qRT-PCR)

MCF-7 Tet-Off empty vector and MCF-7 Tet-Off/ACSL4 total RNA was extracted using Tri-Reagent following the manufacturer's instructions. Any residual genomic DNA was removed by treating RNA with DNase I (15 min at room temperature), which was subsequently inactivated by incubation with 2.5 mM EDTA for 10 min at 65° C. Two μg of total RNA were reverse transcribed using random hexamers and M-MLV Reverse Transcriptase according to the manufacturer's protocol.

For Real-Time PCR, gene specific primers were obtained from RealTimePrimers.com (Elkins Park, Pa., USA). Real-time PCR was performed using Applied Biosystems 7300 Real-Time PCR System. For each reaction, 20 μl of solution containing 5 μl of cDNA, 10 μM forward and reverse primers, and 10 μl of SYBR Select Master Mix was used. All reactions were performed in triplicate. Amplification was initiated by a 2-min preincubation at 50° C., 2-min incubation at 95° C., followed by 40 cycles at 95° C. for 15 sec, 55° C. for 15 sec and 72° C. for 1 min, terminating at 95° C. for the last 15 sec. Gene mRNA expression levels were normalized to human 18S RNA expression, performed in parallel as endogenous control. Real-time PCR data were analyzed by calculating the 2^{-ΔΔct} value (comparative Ct method) for each experimental sample.

Cell proliferation assays were measured by the MTT and 5-bromo-2′-deoxyuridine (BrdU) incorporation as previously described (Maloberti P. M. et al., 2010).

For all the experimental tests carried out in the present invention, data analysis was performed using GraphPad InStat Software 3.01 (La Jolla, Calif., USA). Statistical significance was determined by analysis of variance (ANOVA) followed by Tukey's test.

Wound-Healing Assay

Cellular migration was measured by the wound healing assay, as previously described (Larkins T L et al. Inhibition of cyclooxygenase-2 decreases breast cancer cell motility, invasion and matrix metalloproteinase expression. BMC Cancer. 2006; 6:181). Cells (7×10⁵ cells per well) were seeded in six-well plates. Stable-transfectants were serum-starved for 24 h after which media was replaced (10% FBS medium) and the wound performed. Cells were kept in complete (10% FBS) medium at all times. Wound infliction was considered as time 0 and wound closure was monitored for up to 24 h. Cell monolayer was wounded with a plastic tip across the monolayer cells. Wound closures were photographed by a phase contrast microscopy (40×) at different time points (4, 6, 8, 12 and 24 h) after scraping. The width of the wound was determined with the program Image Pro-Plus.

### Assays

The following assays were carried out in order to verify the inventors' hypothesis as regards the biological mechanism underlying the action of ACSL4 inhibition in connection with tumor cells growth in combination with chemotherapeutic agents.

### Assay 1—Identification of Significantly Up-Regulated Protein Expression of Multidrug Resistance Genes in ACSL4-Overexpressing Cells

For assessing the relationship between the ACSL4 pathway and multidrug resistance genes, the MCF-7/Tet-off/ACSL4 model was used. To this end, protein expression of the multidrug resistance genes ABCG2, ABCC4 and ABCC8 was analyzed in ACSL4-overexpressing cells using Western blot analysis.

These ACSL4-responsive genes were selected among twelve members of the drug resistance genes from the ATP-binding cassette (ABC) family, which were significantly and differentially expressed in MCF-7 Tet-Off/ACSL4 compared to MCF-7 Tet-Off empty vector cells (see Table 1 below).

TABLE 1
Identification of drug resistance genes significantly upregulated by ACSL4 in
MCF-7 Tet-Off/ACSL4 cells through transcriptome analysis

| | | | log2 |
|---|---|---|---|
| Gene Name | Symbol | Location | fold change |
| ATP-Binding Cassette, Sub-Family C (CFTR/MRP) Member 8 | ABCC8 | plasma membrane | 2.952 |
| ATP-Binding Cassette, Sub-Family C (CFTR/MRP), Member 4 | ABCC4 | plasma membrane | 2.235 |
| ATP-Binding Cassette, Sub-Family A (ABC1), Member 12 | ABCA12 | plasma membrane | 2.165 |
| ATP-Binding Cassette, Sub-Family G (WHITE), Member 2 | ABCG2 | mitochondrion/nucleus/plasma membrane | 1.891 |
| ATT-Binding Cassette, Sub-Family B (MDR/TAP), Member 8 | ABCB8 | mitochondrion/nucleus/plasma membrane | 1.879 |
| ATP-Binding Cassette, Sub-Family A (ABC1), Member 7 | ABCA7 | plasma membrane/endosome/golgy apparatus | 1.865 |
| ATP-Binding Cassette, Sub-Family B (MDR/TAP), Member 10 | ABCB10 | mitochondrion | 1.679 |
| ATP-Binding Cassette, Sub-Family A (ABC1), Member 2 | ABCA2 | plasma membrane/endosome/lysosome/vacuole/cytoskeleton | 1.669 |
| ATP-Binding Cassette, Sub-Family F (GCN20), Member 2 | ABCF2 | plasma membrane/mitochondrion | 1.664 |
| ATP-Binding Cassette, Sub-Family B (MDR/TAP), Member 7 | ABCB7 | mitochondrion | 1.647 |
| ATP-Binding Cassette, Sub-Family C (CFTR/MRP), Member 5 | ABCC5 | plasma membrane | 1.621 |
| ATP-Binding Cassette, Sub-Family B (MDR/TAP), Member 9 | ABCB9 | endoplasmic reticulum/lysosome/vacuole | 1.602 |

The human breast cancer resistance protein (originally named BCRP, later renamed as ABCG2) is a member of the G subfamily of the large ATP-binding cassette (ABC) transporter superfamily. ABC proteins transport various molecules across extra- and intracellular membranes. BCRP was initially identified in breast cancer cell lines showing resistance to chemotherapeutic agents.

The ABCC8 protein is also a member of the superfamily of ABC transporters, in particular, of the MRP subfamily which is involved in multi-drug resistance.

In turn, the ABCC4 protein is a member of the ABC1 subfamily. It is apparently involved not only in the efflux of drugs but also in moving nutrients and other biologically important molecules into, out of, and across plasma membranes and intracellular membranes in cells.

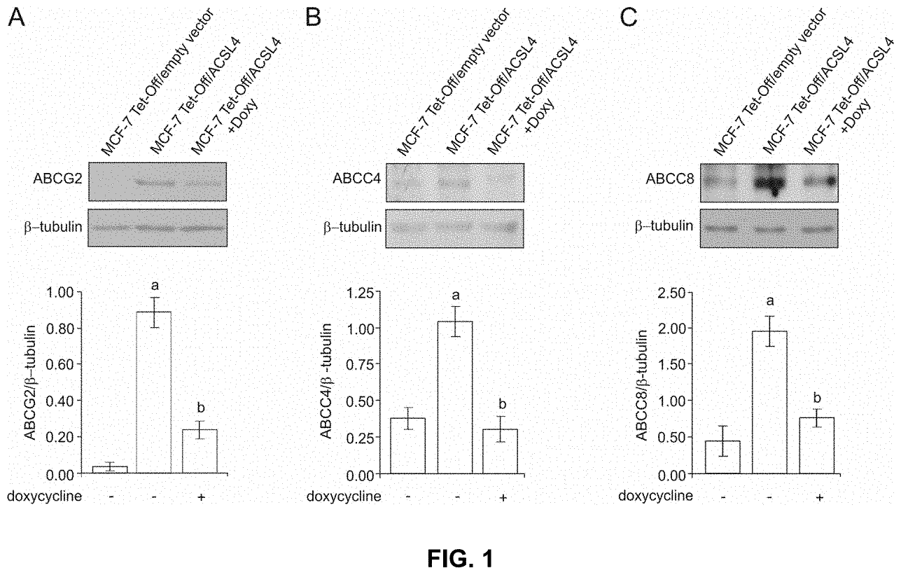

Expression of the selected multidrug resistance proteins (ABCG2, ABCC4 and ABCC8) was identified in the MCF-7/Tet-Off system, by Western blot analysis, using tubulin as a loading control. FIG. 1 shows the integrated optical density levels of each protein normalized with the β-tubulin signal (upper images). Also, representative blots for each group of cells (MCF-7/Tet-Off empty vector, MCF-7/Tet-Off ACSL4 and MCF-7/Tet-Off ACSL4+doxycycline) as fold change, are shown for A. ABCG2 protein, B. ABCC4 protein and C. ABCC8 protein.

By means of the present assay, it is shown that expression of ACSL4 increases the expression levels of these three drug resistance proteins. As may be seen, when the ACSL4-expressing cells were incubated in the presence of doxycycline (a specific ACSL4 expression inhibitor in the Tet-Off system) the increase in ABCG2, ABCC4 and ABCC8 is inhibited and lowered to basal levels, thus showing the specificity in the response by ACSL4 expression.

In the assay performed herein, the present inventors demonstrated that ACSL4 expression increased the protein expression levels of the mentioned proteins. Also, the test shows the specificity in the response by ACSL4 expression given that when the ACSL4-expressing cells were incubated in the presence of doxycycline (a specific ACSL4 expression inhibitor) the increase in protein expression is inhibited and lowered to basal levels, thus confirming the specific role of ACSL4 in the regulation on the energy-dependent transporter proteins expression.

### Assay 2—Effect of ACSL4 Inhibition on Energy-Dependent Transporters in MDA-MB-231 Cells

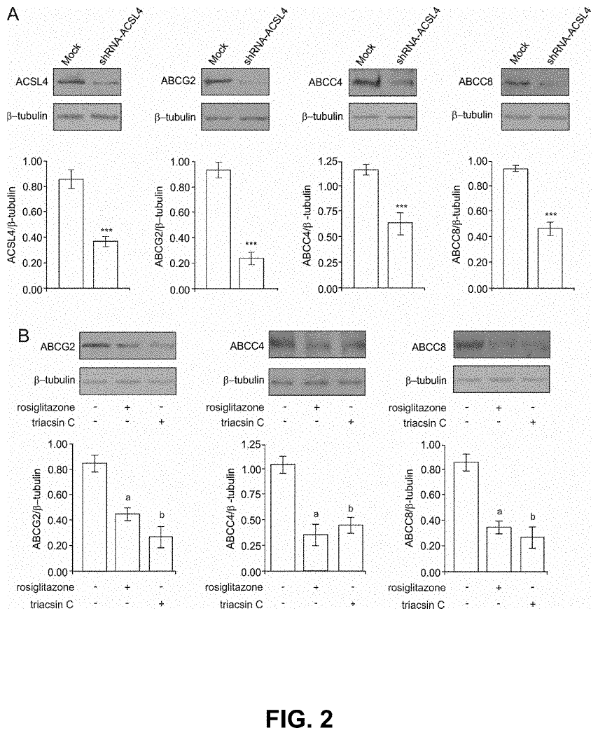

Given that overexpression experiments succeeded in forcing ACSL4 action and to further validate the results described above through a control experiment, we next disrupted the expression of endogenous ACSL4 using small hairpin RNA (shRNA) in the highly aggressive MD-MB-231 breast cancer cells, which constitutively overexpress ACSL4. Western blot analysis showed that the shRNA-targeting ACSL4 markedly decreased the expression of ACSL4 protein as previously described [Maloberti et al., 2010, supra], while ACSL4 knock-down sharply decreased the protein levels of ABCG2, ABCC8 and ABCC4 (FIG. 2 A). In addition, using the same cell line as described above, we inhibited the activity of endogenous ACSL4 using triacsin C and rosiglitazone, two well-known inhibitors of ACSL4 activity (Orlando et al., 2015; Askari B, Kanter J E, Sherrid A M Golej D L, Bender A T, Liu J, Hsueh W A, Beavo J A, Coleman R A and Bornfeldt K E. Rosiglitazone inhibits acyl-CoA synthetase activity and fatty acid partitioning to diacylglycerol and triacylglycerol via a peroxisome proliferator-activated receptor-gamma-independent mechanism in human arterial smooth muscle cells and macrophages. Diabetes. 2007; 56(4): 1143-1152; Kim J H, Lewin T M and Coleman R A. Expression and characterization of recombinant rat Acyl-CoA synthetases 1, 4, and 5. Selective inhibition by triacsin C and thiazolidinediones. J Biol Chem. 2001; 276(27):24667-24673). Western blot analysis showed that the inhibitors targeting ACSL4 activity also markedly decreased the protein levels of ABCG2, ABCC8 and ABCC4 (FIG. 2 B). Altogether, these results confirmed those obtained by transcriptome analysis showing that ACSL4 expression regulates the expression of proteins involved in resistance to anti-cancer drug treatment.

### Transfection of MDA-MB-231

MDA-MB-231 cells were seeded the day before and grown up to 80% confluence. Transfection was performed in Opti-MEM medium with Lipofectamine 2000 reagent (Invitrogen) using the pSUPER.retro plasmid (OligoEngine, Seattle, Wash., USA) containing ACSL4 shRNA (AAGATTATTCTGTGGATGA) (SEQ ID NO: 1). The empty vector was used as control. Transfection efficiency was estimated by counting fluorescent cells transfected with the pRc/CMVi plasmid containing the enhanced form of green fluorescent protein [Maloberti et al.; 2010]. Forty-eight hours after transfection, cells were selected in media containing 1 μg/ml Puromycin for 1 month and then collected for biochemical and cellular assays. MDA-MB-231 transfected cell lines were designated MDA-MB-231 shRNA-ACSL4, and MDA-MB-231 mock, respectively. One month post selection, whole cell extracts were obtained and analyzed by Western blot as previously described [Maloberti et al.; 2010] using the indicated antibodies.

Assay 3—Determination of the Effect of Chemotherapeutic Agents in the Context of ACSL4-Expressing Cells with and without ACSL4 Inhibition

The effect of two exemplary chemotherapeutic agents: doxorubicin and paclitaxel, was analyzed using the MCF-7 Tet-Off system.

This well known system is based on the Tetracycline-controlled transcriptional activation of an artificially-inserted gene, in this case, the ACSL4 gene. The tetracycline Tet-Off system is used to stably transfect non-aggressive breast cancer MCF-7 cells to develop a stable line overexpressing ACSL4 (MCF-7 Tet-Off/ACSL4). In turn, those cells not transfected with the ACSL4 cDNA are called MCF-7 Tet-Off empty vector.

Separate experiments were carried out for each one of the tested chemotherapeutic agents. Each chemotherapeutic agent was first tested alone at the concentrations used, and then in combination with doxycycline, for each of the cell groups: MCF-7 Tet-Off/ACSL4 cells, MCF-7 Tet-Off empty vector cells and doxycycline-treated MCF-7 Tet-Off/ACSL4 cells, the latter is used to specifically override ACSL4 expression.

### 3.1—Doxorubicin Effect in ACSL4-Expressing and ACSL4 Non-Expressing Cells

MCF-7 Tet-Off empty vector and MCF-7 Tet-Off/ACSL4 cells were plated at a density of 4000 cells/well in 96-well plates with 10% FBS-supplemented D-MEM medium and allowed to adhere overnight at 37° C. in a humidified, 5% CO₂ atmosphere. The medium was then changed to serum-free medium. After 24 h, the cells were switched to 10% FBS-supplemented D-MEM medium and incubated with doxorubicin at varying (sub-therapeutically effective or non-effective per se) concentrations (0.025, 0.050, 0.100 μM) for 96 h. Subsequently, cell proliferation was measured by the bromo-deoxyuridine (BrdU) incorporation assay.

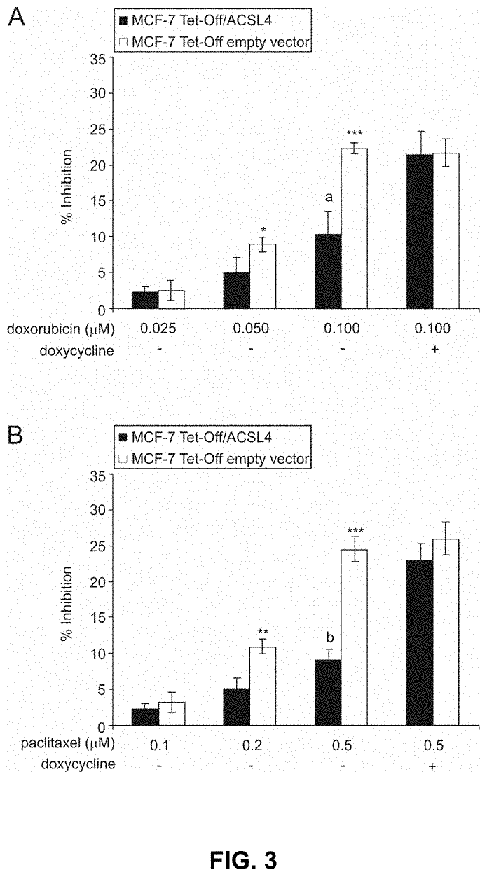

Treatment with the used doses of doxorubicin had very little or no effect on the inhibition of MCF-7 Tet-Off/ACSL4 cells proliferation, while it inhibited proliferation of the MCF-7 Tet-Off empty vector cells. In turn, as can be seen in FIG. 3A, doxorubicin in doxycycline-treated cells produced a significantly increased inhibition of cell proliferation, thus showing that inhibition of ACSL4 in the cancer cell line enhances the effect of the chemotherapeutic agent.

### 3.2—Paclitaxel Effect in ACSL4-Expressing and ACSL4 Non-Expressing Cells

MCF-7 Tet-Off empty vector and MCF-7 Tet-Off/ACSL4 cells were plated at a density of 4000 cells/well in 96-well plates with 10% FBS-supplemented D-MEM medium and allowed to adhere overnight at 37° C. in a humidified, 5% CO₂ atmosphere. The medium was then changed to serum-free medium. After 24 h, the cells were switched to 10% FBS-supplemented D-MEM medium and incubated with paclitaxel at varying (sub-therapeutically effective or non-effective per se) concentrations (0.1, 0.2, 0.5 μM) for 96 h. Subsequently, cell proliferation was measured by the bromo-deoxyuridine (BrdU) incorporation assay.

Treatment with the used doses of paclitaxel had little or no effect on the inhibition of MCF-7 Tet-Off/ACSL4 cells proliferation, while it inhibited proliferation of the MCF-7 Tet-Off empty vector cells. In turn, as can be seen in FIG. 3B, paclitaxel in doxycycline-treated cells produced a significantly increased inhibition of cell proliferation, thus showing that inhibition of ACSL4 in the cancer cell line enhances the effect of the chemotherapeutic agent.

### EXAMPLES

The invention is further illustrated by the following Examples, which are not intended to limit the scope thereof. Instead, the examples set forth below should be understood only as exemplary embodiments for better taking into practice the present invention.

### Example 1

### Rosiglitazone and Doxorubicin Combination for Inhibiting Cell Proliferation

To further analyze pathway sequential activation, the effect of doxorubicin was also tested both alone and in combination with rosiglitazone—an inhibitor of ACSL4—on MDA-MB-231 cells, a triple negative breast cancer cell line.

MBA-MB-231 cells were plated at a density of 4000 cells/well in 96-well plates with 10% FBS-supplemented D-MEM medium and allowed to adhere overnight at 37° C. in a humidified, 5% CO₂ atmosphere. The medium was then changed to serum-free medium. After 24 h, the cells were switched to 10% FBS-supplemented D-MEM medium and incubated with doxorubicin and/or rosiglitazone for 96 h. Subsequently, cell proliferation was measured by the bromo-deoxyuridine (BrdU) incorporation assay.

Rosiglitazone and doxorubicin were first tested alone at the concentrations used, and then in combination.

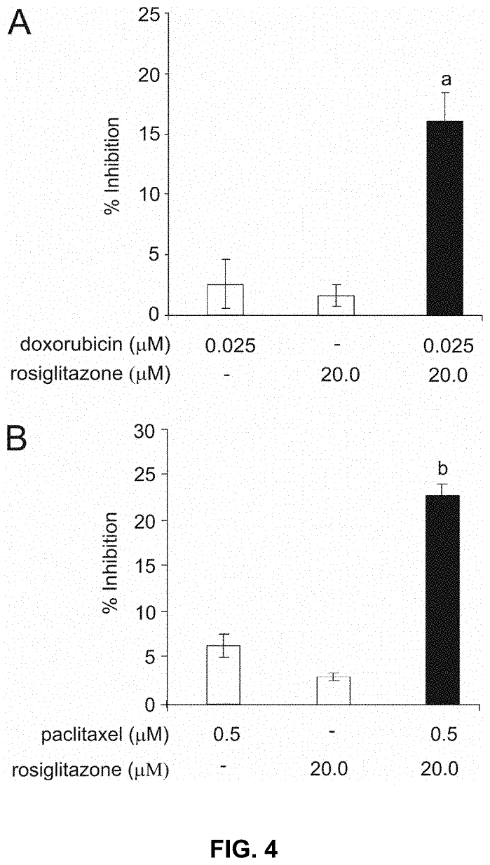

The combination treatment with non-effective or sub-therapeutically effective doses of doxorubicin and rosiglitazone showed a significant inhibition of MDA-MB-231 cell proliferation (FIG. 4A). At the doses used, doxorubicin and rosiglitazone—each individually—had very little effect on the proliferation. In turn, doxorubicin and rosiglitazone together produced a significantly increased effect in the inhibition of cell proliferation.

Based on these results, ACSL4 turns out to be a potential therapeutic target, the inhibitors of which can be used in combination with chemotherapeutic agents, thus preventing the side effects of the long-term therapeutic doses thereof (Gelmon K et al., Targeting triple-negative breast cancer: optimising therapeutic outcomes. Ann Oncol. 2012 Sep; 23(9):2223-34; Yardley D. A., Drug Resistance and the Role of Combination Chemotherapy in Improving Patient Outcomes. International Journal of Breast Cancer 2013; 2013:1-15) and generate more positive effects than single-drug therapy.

As can be seen, the used doses of rosiglitazone in combination with doxorubicin, produce a significant inhibition of cell proliferation, thus preventing the adverse side effects of therapeutic doses of each active ingredient.

### Example 2

### Rosiglitazone and Paclitaxel Combination for Inhibiting Cell Proliferation

The effect of paclitaxel was also tested both alone and in combination with rosiglitazone—an inhibitor of ACSL4—on MDA-MB-231 cells.

MDA-MB-231 cells were plated at a density of 4000 cells/well in 96-well plates with 10% FBS-supplemented D-MEM medium and allowed to adhere overnight at 37° C. in a humidified, 5% CO₂ atmosphere. The medium was then changed to serum-free medium. After 24 h, the cells were switched to 10% FBS-supplemented D-MEM medium and incubated with paclitaxel and/or rosiglitazone for 96 h. Subsequently, cell proliferation was measured by the bromo-deoxyuridine (BrdU) incorporation assay.

Rosiglitazone and paclitaxel were first tested alone at the concentrations used, and then in combination.

In combination treatment, non-effective or sub-therapeutically effective doses of paclitaxel and rosiglitazone showed a significant inhibition of MDA-MB-231 cell proliferation (FIG. 4B). At the doses used, paclitaxel and rosiglitazone—each individually—had very little effect on the proliferation. In turn, paclitaxel and rosiglitazone together produced a significantly increased effect in the inhibition of cell proliferation.

On the basis of these results, ACSL4 is a potential therapeutic target. The present experiment suggests that ACSL4 inhibitors can be used in combination with chemotherapeutic agents, thus preventing the side effects of the long-term therapeutic doses thereof (Gelman K et al., 2012; Yardley D. A., 2013) and generate more positive effects than single-drug therapy.

As can be seen, the used doses of rosiglitazone in combination with paclitaxel, produce a significant inhibition of cell proliferation, thus preventing the adverse side effects of individual therapeutic doses.

### Example 3

### Triacsin C and Doxorubicin Combination for Inhibiting Cell Proliferation

Using the same procedure as that described in Example 1, the effect of doxorubicin was also tested on MDA-MB-231 cells, both alone and in combination with Triacsin C, another ACSL4 inhibitor, non-structurally related with rosiglitazone.

MBA-MB-231 cells were plated at a density of 4000 cells/well in 96-well plates with 10% FBS-supplemented D-MEM medium and allowed to adhere overnight at 37° C. in a humidified, 5% CO₂ atmosphere. The medium was then changed to serum-free medium. After 24 h, the cells were switched to 10% FBS-supplemented D-MEM medium and incubated with doxorubicin and/or Triacsin C for 96 h. Subsequently, cell proliferation was measured by the bromo-deoxyuridine (BrdU) incorporation assay.

Cell proliferation % was measured by testing Triacsin C and doxorubicin each one alone first, at the concentrations used, and then in combination.

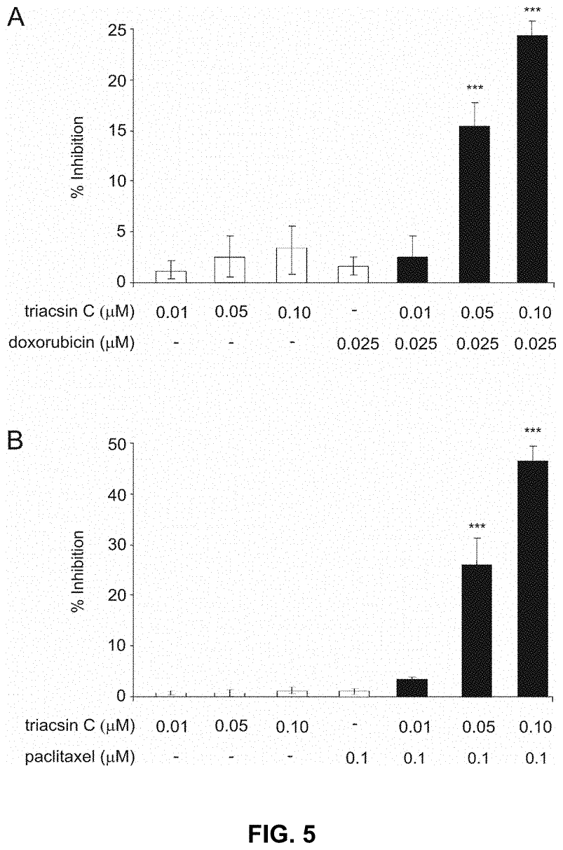

The combined treatment with non-effective or sub-therapeutically effective doses of doxorubicin and Triacsin C showed a significant inhibition of MDA-MB-231 cell proliferation (FIG. 5A). In turn, doxorubicin and Triacsin C together produced a significantly increased effect in the inhibition of cell proliferation. At the doses used, doxorubicin and Triacsin C—each individually—had very little effect on the proliferation.

It may be concluded that ACSL4 inhibitors can be used in combination with chemotherapeutic agents, thus preventing the adverse effects of the long-term therapeutic doses thereof and generate more positive effects than single-drug therapy.

### Example 4

### Triacsin C and Paclitaxel Combination for Inhibiting Cell Proliferation

Using the same procedure as that described in Example 2, the effect of paclitaxel was also tested both alone and in combination with Triacsin C, another ACSL4 inhibitor, non-structurally related with rosiglitazone.

MDA-MB-231 cells were plated at a density of 4000 cells/well in 96-well plates with 10% FBS-supplemented D-MEM medium and allowed to adhere overnight at 37° C. in a humidified, 5% CO₂ atmosphere. The medium was then changed to serum-free medium. After 24 h, the cells were switched to 10% FBS-supplemented D-MEM medium and incubated with paclitaxel and/or Triacsin C for 96 h. Subsequently, cell proliferation was measured by the bromo-deoxyuridine (BrdU) incorporation assay.

Triacsin C and paclitaxel were first tested alone at the concentrations used, and then in combination.

Combined treatment with non-effective or sub-therapeutically effective doses of paclitaxel and Triacsin C showed a significant inhibition of MDA-MB-231 cell proliferation (FIG. 5B). In turn, paclitaxel and Triacsin C together produced a significantly increased effect in the inhibition of cell proliferation. At the doses used, paclitaxel and Triacsin C—each individually—had very little effect on the proliferation.

On the basis of these results, ACSL4 is a potential therapeutic target. The present experiment suggests that ACSL4 inhibitors can be used in combination with chemotherapeutic agents, thus preventing the side effects of the long-term therapeutic doses thereof and generate more positive effects than single-drug therapy.

As can be seen, the used doses of Triacsin C in combination with paclitaxel, produce a significant inhibition of cell proliferation, thus preventing the side effects of therapeutic doses.

### Example 5

### Inhibition of Cell Migration Through the Combination of Sub-Effective Doses of Rosiglitazone and Doxorubicin or Paclitaxel

In order to validate a potential clinical significance of the novel regulatory effect of ACSL4 on energy-dependent transporter expression, we used a combined pharmacological approach in order to observe the efficacy of ACSL4 inhibition in combination with the chemotherapeutic agents to reverse drug resistance. To this end, we tested therapeutic efficacy to inhibit cell migration through sub-maximal doses of the chemotherapeutic drug doxorubicin combined with rosiglitazone, a well-known inhibitor of ACSL4 activity.

Inhibition on cell migration was assessed by combining sub-effective doses of ACSL4 inhibitor rosiglitazone and chemotherapeutic agents in MDA MB-231 cells. Cellular migration was measured by the wound healing assay as follows: cells (7×10⁵ cells per well) were seeded in six-well plates. Stable-transfectants were serum-starved for 24 h after which media was replaced (10% FBS medium) and the wound performed. Cells were kept in complete (10% FBS) medium at all times. Wound infliction was considered as time 0 and wound closure was monitored for up to 24 h. Cell monolayer was wounded with a plastic tip across the monolayer cells. Wound closures were photographed by a phase contrast microscopy (40×) at different time points (4, 6, 8, 12 and 24 h) after scraping. The width of the wound was determined with the program Image Pro-Plus.

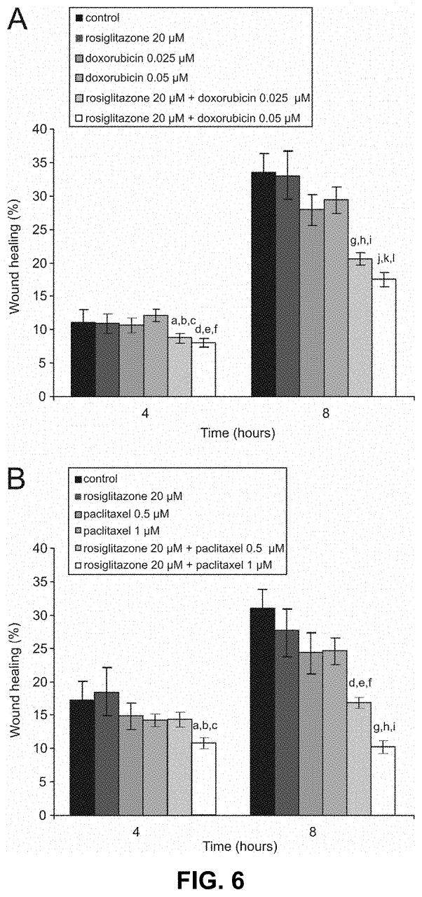

Cells were incubated with rosiglitazone (20 μM) and/or A. doxorubicin (0.025 and 0.05 μM), B. paclitaxel (0.5 and 1.0 μM). At the specified time points, the distance between the wound edges was measured using Image-Pro Plus software (see FIG. 6).

Surprisingly, the same results as those for the previous analysis were obtained in these migration inhibition tests performed through wound healing assays.

### Example 6

### Nude Mouse Xenograft Model—In Vivo Therapy of Solid Tumors

The experimental design for the present example followed a well-established female nude mouse model [Perera Y, Farina H G, Hernandez I, Mendoza O, Serrano J M, Reyes O, Gomez D E, Gomez R E, Acevedo B E, Alonso D F and Perea S E. Systemic administration of a peptide that impairs the protein kinase (CK2) phosphorylation reduces solid tumor growth in mice. Int j Cancer. 2008].

MDA-MB-231 cells (5×10⁶ cells) mixed with Matrigel Matrix (BD Biosciences) were injected into the right flank of female Foxn1 nu/nu Balb/c athymic nude mice, aged 6-8 weeks, and allowed to form tumors. Tumors were measured with calipers every other day (length and width) and the mice weighed. Mice were provided with free access to food, water and bedding at all time and were housed with a 12 h light/dark cycle in filter top cages containing a maximum of six mice per cage. Tumor volumes (mm³) were calculated by the formula: π/6×width²(mm²)×length (mm) as described previously [Orlando et.al., 2012; Perera Y et.al. 2008]. The experiment was terminated as previously described [Orlando et al. 2012; Ripoll G V, Giron S, Krzymuski M J, Hermo G A, Gomez D E and Alonso D E Antitumor effects of desmopressin in combination with chemotherapeutic agents in a mouse model of breast cancer. Anticancer Res. 2008] in accordance with institutionally approved guidelines.

Given our previous in vitro results and the demonstration that modulating ACSL4 inhibition results in the downregulation of energy-dependent transporter expression with a consequent change in cell phenotype and sensitivity to drugs, the next step was to analyze the effect of ACSL4 inhibitor and chemotherapeutic agents on tumor growth in vivo. The MDA-MB-231 cell line is known to form tumors with a triple-negative signature which do not respond to hormone treatment and are highly resistant to radio o chemotherapy. This has led to extensive use of the MDA-MB-231 xenograft model to test treatment efficacy. In particular, MDA-MB-231 is a natural model to investigate whether a combinatorial therapy targeting ACSL4 is effective in reducing tumor growth.

Following a 4-day window to allow the establishment of tumor xenografts, mice injected with tumor cells were randomly separated into groups and daily treated with the ACSL4 inhibitor rosiglitazone and the chemotherapeutic agent doxorubicin alone or in a combination, or appropriate controls. Treated mice were sacrificed on day 30 post tumor cell injection and tumor samples were collected as previously described [Orlando et al., 2012].

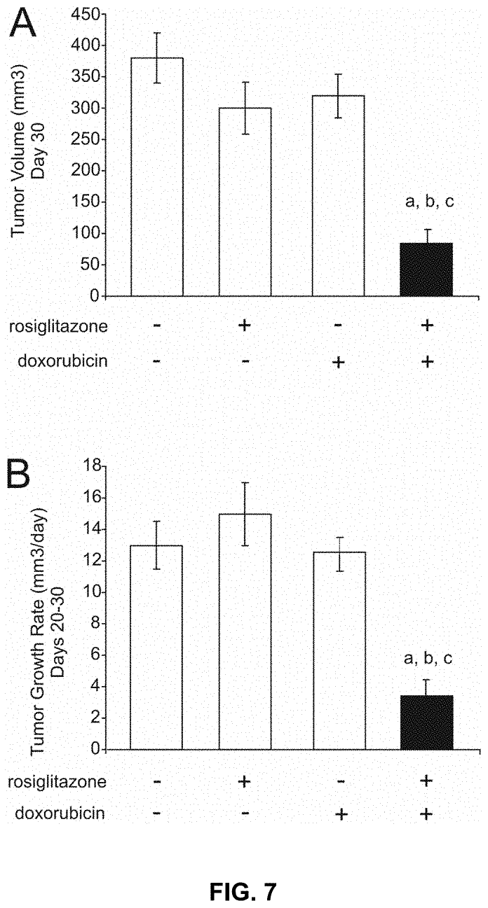

Although the MDA-MB-231 xenograft growth rate varies among studies reported in the literature, our tumor xenografts were in the range of those previously reported [Orlando et al., 2012; Orlando et al., 2015]. The average animal body weight was 23.5 g at the beginning of the treatment, and no significant differences in body weight were observed between the different treatment groups at the end of the experiment. The amount of food intake in the control group compared to the treated groups was not significantly different throughout the experiment. However, as shown in FIGS. 7A and 7B, there was a significant inhibition in tumor volume and growth rate in mice subjected to combination therapy using doxorubicin, as compared to those receiving single drug-based treatments or drug vehicle after injection of MDA-MB-231 cells. Interestingly, the compounds assayed markedly reduced tumor volume and growth rate at concentrations which are ineffective when used alone. These results point to a synergistic effect, with the key advantage of exposing mice to lower drug concentrations.

The experimental design for the MDA-MB231 xenograft model used in the present example was carried out in strict accordance with the recommendations in the Guide for the Care and Use of Laboratory Animals of the National Institutes of Health. The protocol was approved by the Institutional Ethical Committee from the School of Medicine, University of Buenos Aires (ID: 093/10 CD, School of Medicine).

1. A method for treating a patient having a tumor overexpressing ACSL4, the method comprising administering to the patient: i) a dosage form of a first component being an ACSL4 inhibitor selected from the group of Triacsin C and rosiglitazone; and ii) a dosage form of a second component being a chemotherapeutic agent selected from doxorubicin, paclitaxel and docetaxel, wherein the tumor is colon carcinoma, hepatocellular carcinoma, prostate cancer, breast cancer or triple negative breast cancer (TNBC).

2. The method according to claim 1, wherein the ACSL4 inhibitor and the chemotherapeutic agent are present in sub-therapeutically effective amounts.

3. The method according to claim 1, wherein the tumor is metastatic.

4. The method according to claim 1, wherein the triple negative breast cancer (TNBC) is metastatic.

5. The method according to claim 1, wherein the ACSL4 inhibitor is Triacsin C, and the chemotherapeutic agent is selected from paclitaxel and docetaxel.

6. The method according to claim 1, wherein the ACSL4 inhibitor is Triacsin C, and the chemotherapeutic agent is doxorubicin.

7. The method according to claim 1, wherein the ACSL4 inhibitor is rosiglitazone, and the chemotherapeutic agent is selected from paclitaxel and docetaxel.

8. The method according to claim 1, wherein the ACSL4 inhibitor is rosiglitazone, and the chemotherapeutic agent is doxorubicin.

9. The method according to claim 1, wherein the ACSL4 inhibitor is administered prior, or during administering the chemotherapeutic agent.


SEQUENCE LISTING (1 sequence)

SEQ ID NO: 1 - DNA, 19 bp, Artificial Sequence
  ACSL4 shRNA. Synthetic sequence designed to knock down ACSL4 expression.
aagattattctgtggatga